(19) United States
(12) Patent Application Publication
(10) Pub. No.: US 2025/0082684 A1
(43) Pub. Date: Mar. 13, 2025

(54) Recombinant Cell Line Expressing Membrane Proteins and Vesicles Prepared Therefrom

(71) Applicant: Exocure Sweden AB, Göteborg (SE)

(72) Inventors: Jan Lötvall, Göteborg (SE); Kyong-su Park, Göteborg (SE); Cecilia Lasser, Göteborg (SE)

(21) Appl. No.: 18/728,983

(22) PCT Filed: Jan. 17, 2023
(86) PCT No.: PCT/US2023/010951

§ 371 Date: Jul. 15, 2024

Related U.S. Application Data
(60) Provisional application No. 63/301,233, filed on Jan. 20, 2022.

(51) Int. Cl. A61K 35/28; A61P 37/06; C12N 5/0775

(52) U.S. Cl. CPC A61K 35/28; A61P 37/06; C12N 5/0662; C12N 2510/00

(57) ABSTRACT

The present disclosure provides vesicles prepared from a mammalian cell line genetically modified to overexpress one or more membrane proteins as provided in the disclosure. The genetically modified cells are also provided. The vesicles find use in methods for reducing inflammatory response in a subject in need thereof.

### CROSS-REFERENCE TO RELATED APPLICATIONS

This application claims priority to U.S. Provisional Application No. 63/301,233 filed on Jan. 20, 2022 which application is herein incorporated by reference in its entirety.

### INTRODUCTION

Vesicles, including extracellular vesicles (EVs) or microvesicles (MVs), nanovesicles (NVs), and synthetic eukaryotic vesicles (SyEVs), have been described. These vesicles, when produced by mesenchymal stem cells or other stem cells, have been used as anti-inflammatory agents. However, it is not known which molecule(s) present in these vesicles are required for their anti-inflammatory activity. Such a molecule(s) can be used to generate vesicles from cells that have been genetically modified to overexpress the molecule(s).

The present disclosure provides cells and vesicles from the cells that have been genetically modified to overexpress membrane proteins associated with the anti-inflammatory activity of EVs, NVs, and SyEVs.

### SUMMARY

The present disclosure provides vesicles prepared from a mammalian cell line genetically modified to overexpress a membrane protein selected from the group consisting of: bifunctional heparan sulfate N-deacetylase/N-sulfotransferase 1, ferroptosis suppressor protein 1, cysteine-rich and transmembrane domain-containing protein, cadherin-6, solute carrier family 22 member 18, promethin, receptor-type tyrosine-protein phosphatase kappa, prolyl endopeptidase FAP, phospholipid-transporting ATPase ID, ADP-ribosyl cyclase/cyclic ADP-ribose hydrolase 2, reticulon-2, ras-related protein Rap-1A, basigin, aldehyde dehydrogenase family 3 member B1, calsyntenin-1, small VCP/p97-interacting protein, GTP-binding protein Rheb, lysophosphatidic acid receptor 1, prolow-density lipoprotein receptor-related protein 1, receptor-type tyrosine-protein phosphatase F, raftlin, guanine nucleotide-binding protein G (q) subunit alpha, guanine nucleotide-binding protein subunit alpha-11, integrin alpha-8, roundabout homolog 1, ADP-ribosylation factor 6, ephrin type-B receptor 2, transforming protein RhoA, proto-oncogene tyrosine-protein kinase Src, synaptobrevin homolog YKT6, Ras-related protein Rab-23, vascular cell adhesion protein 1, lymphocyte function-associated antigen 3, guanine nucleotide-binding protein G (I)/G(S)/G (O) subunit gamma-12, 2′,3′-cyclic-nucleotide 3′-phosphodiesterase, receptor-type tyrosine-protein phosphatase eta, guanine nucleotide-binding protein G(s) subunit alpha isoforms short, ADP-ribosylation factor 5, mitochondrial calcium uniporter regulator 1, nuclear mitotic apparatus protein 1, protein/nucleic acid deglycase DJ-1, V-type immunoglobulin domain-containing suppressor of T-cell activation, CD276, MCAM and Phosphatidylserine synthase 2. The genetically modified cells are also provided. The vesicles find use in methods for reducing inflammatory response in a subject in need thereof.

### BRIEF DESCRIPTION OF DRAWINGS

FIG. 1 is a schematic diagram showing the isolation method of extracellular vesicles (EVs), nanovesicles (NVs), and synthetic eukaryotic vesicles (SyEVs) from mesenchymal stem cells (MSCs).

FIG. 2 is a graph showing reduction in OMV-induced pro-inflammatory cytokines by EVs, NVs or SyEVs. OMV (100 ng/ml) were added to the RAW 264.7 cells for 3 hours, followed by washing with PBS, and treatment of EVs, NVs or SyEVs (10⁷, 10⁸, 10⁹). Supernatant concentrations of TNF-α (A) and IL-6 (B) were measured by ELISA. **, P<0.01; ***, P<0.001; two way ANOVA with Tukey's multiple comparison test. Error bars indicate SEM. N=3.

FIG. 3 shows Venn diagram of the 605 proteins associated with the Gene ontology term “integral component of membrane”. Proteins with a fold change above 1.5 was considered up-regulated in each comparison. The proteins with a fold change below 1.5 was considered none-altered between the two vesicles compared.

FIG. 4 shows Venn diagram of the proteins considered to be none-altered between the vesicles types in FIG. 3.

FIGS. 5A and 5B show the list of proteins which are considered non-altered in all three comparisons in FIG. 4.

### Definitions

The term “vesicle” as used herein refers to a spherical structure which contains an interior volume that is separated from the outside environment by a lipid bilayer membrane. A vesicle can be secreted from cells or can be artificially synthesized from a cell. A vesicle is generally smaller than the cell from which it is derived.

The term “outer membrane vesicle(s)” or “OMV(s)” as used herein refers to vesicles that include an outer membrane enclosing periplasmic contents, cytoplasmic contents and inner membrane components. OMVs include blebs produced by budding of the outer membrane of organisms, such as, gram-negative bacteria. Such OMVs can also be referred to as native OMVs. OMVs can also be produced by disrupting (e.g., by extrusion, sonication, detergents, or osmotic shock) a gram-negative bacterium in a hydrophilic solution thereby forcing the cell to form vesicles.

The term “revesiculation” and grammatical equivalents thereof, as used herein refers to a process of opening a vesicle, e.g., a cell-derived vesicle, such that the interior contents of the vesicle are released, followed by isolation of the open lipid bilayer membrane, and closing of the open lipid bilayer membrane to reform vesicles. Such vesicles are referred to as ghost vesicles (gNVs) or synthetic eukaryotic vesicles (SyEVs).

The term “non-revesiculated” and grammatical equivalents thereof, as used herein refers to a vesicle, e.g., a cell-derived vesicle that is not a ghost vesicle, i.e., has not been subjected to the process of opening the vesicle such that the interior contents of the vesicle are released, followed by isolation of the open lipid bilayer membrane, and closing of the open lipid bilayer membrane to reform vesicles. Thus, a non-revesiculated vesicle encloses significantly more of the interior contents from the cell from which it is derived as compared to a ghost vesicle prepared from the same type of cell.

As used herein, the term “extracellular vesicle” means a vesicle released by a eukaryotic, e.g., a mammalian cell. Examples of “extracellular vesicles” include exosomes, ectosomes, microvesicles, prostasomes, oncosomes, and apoptotic bodies.

The term “inflammatory response” as used herein refers to secretion of proinflammatory cytokines, activation of toll-like receptors (TLR) and/or systemic inflammation. Examples of proinflammatory cytokines include IL-6 IL-2, IL-4, IL-6, IL-12, IL-12p70, IL-17, tumor necrosis factor alpha (TNF-α) and interferon gamma (IFN-v).

“Isolated” refers to an entity of interest that is in an environment different from that in which it may naturally occur. “Isolated” is meant to include entities that are within samples that are substantially enriched for the entity of interest and/or in which the entity of interest is partially or substantially purified.

The terms “subject” and “patient” refers to an animal which is the object of treatment, observation, or experiment. By way of example only, a subject includes, but is not limited to, a mammal, including, but not limited to, a human or a non-human mammal, such as a non-human primate, bovine, equine, canine, ovine, or feline.

The terms “treatment,” “treat,” or “treating,” as used herein cover any treatment of a disease or condition of a mammal, particularly a human, and includes: (a) preventing the disease or condition from occurring in a subject which may be predisposed to the disease or condition but has not yet been diagnosed as having it; (b) inhibiting the disease or condition, i.e., arresting its development; (c) relieving and or ameliorating the disease or condition, i.e., causing regression of the disease or condition; or (d) curing the disease or condition, i.e., stopping its development or progression. The population of subjects treated by the methods of the invention includes subjects suffering from the undesirable condition or disease, as well as subjects at risk for development of the condition or disease.

The term “therapeutic effect” refers to some extent of relief of one or more of the symptoms of a disorder (e.g., infection, a neoplasia or tumor) or its associated pathology. “Therapeutically effective amount” as used herein refers to an amount of an agent which is effective, upon single or multiple dose administration to the cell or subject, in prolonging the survivability of the patient with such a disorder, reducing one or more signs or symptoms of the disorder, preventing or delaying, and the like beyond that expected in the absence of such treatment. “Therapeutically effective amount” is intended to qualify the amount required to achieve a therapeutic effect. A physician or veterinarian having ordinary skill in the art can readily determine and prescribe the “therapeutically effective amount” (e.g., ED50) of the pharmaceutical composition required. For example, the physician or veterinarian could start doses of the vesicles of the present disclosure employed in a pharmaceutical composition at levels lower than that required in order to achieve the desired therapeutic effect and gradually increase the dosage until the desired effect is achieved.

“Conservative amino acid substitution” refers to a substitution of one amino acid residue for another sharing chemical and physical properties of the amino acid side chain (e.g., charge, size, hydrophobicity/hydrophilicity). “Conservative substitutions” are intended to include substitution within the following groups of amino acid residues: gly, ala; val, ile, leu; asp, glu; asn, gln; ser, thr; lys, arg; and phe, tyr. Guidance for such substitutions can be drawn from alignments of amino acid sequences of polypeptides presenting the epitope of interest.

The term “heterologous” refers to two biological components that are not found together in nature. The components may be host cells, genes, or regulatory regions, such as promoters. Although the heterologous components are not found together in nature, they can function together, as when a promoter heterologous to a gene is operably linked to the gene.

The term “endogenous” refers to a naturally-occurring biological component of a cell, i.e., as found in nature.

“Recombinant” as used herein refers to nucleic acid encoding a gene product, or a gene product (e.g., polypeptide) encoded by such a nucleic acid, that has been manipulated by the hand of man, and thus is provided in a context or form in which it is not found in nature. “Recombinant” thus encompasses, for example, a nucleic acid encoding a gene product operably linked to a heterologous promoter (such that the construct that provides for expression of the gene product from an operably linked promoter with which the nucleic acid is not found in nature). For example, a “recombinant outer membrane polypeptide” encompasses an outer membrane polypeptide encoded by a construct that provides for expression from a promoter heterologous to the outer membrane polypeptide coding sequence, outer membrane polypeptides that are modified relative to a naturally-occurring outer membrane (e.g., as in a fusion protein), and the like. It should be noted that a recombinant outer membrane polypeptide can be endogenous to or heterologous to a cell in which such a recombinant nucleic acid is present.

The terms “subject” and “patient” refers to an animal which is the object of treatment, observation, or experiment. By way of example only, a subject includes, but is not limited to, a mammal, including, but not limited to, a human or a non-human mammal, such as a non-human primate, bovine, equine, canine, ovine, or feline.

Any cells, agents, vesicles, compositions or methods provided herein can be combined with one or more of any of the other cells, agents, vesicles, compositions and methods provided herein, regardless of whether they are disclosed in separate sections of the application or within the same section of the application.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a vesicle” includes a plurality of such vesicles and reference to “the vesicle” includes reference to one or more vesicles and equivalents thereof known to those skilled in the art and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication. Further, the dates of publication provided may be different from the actual publication dates which may need to be independently confirmed.

### DETAILED DESCRIPTION

The present disclosure provides vesicles prepared from a mammalian cell line genetically modified to overexpress a membrane protein selected from the group consisting of: bifunctional heparan sulfate N-deacetylase/N-sulfotransferase 1, ferroptosis suppressor protein 1, cysteine-rich and transmembrane domain-containing protein, cadherin-6, solute carrier family 22 member 18, promethin, receptor-type tyrosine-protein phosphatase kappa, prolyl endopeptidase FAP, phospholipid-transporting ATPase ID, ADP-ribosyl cyclase/cyclic ADP-ribose hydrolase 2, reticulon-2, ras-related protein Rap-1A, basigin, aldehyde dehydrogenase family 3 member B1, calsyntenin-1, small VCP/p97-interacting protein, GTP-binding protein Rheb, lysophosphatidic acid receptor 1, prolow-density lipoprotein receptor-related protein 1, receptor-type tyrosine-protein phosphatase F, raftlin, guanine nucleotide-binding protein G (q) subunit alpha, guanine nucleotide-binding protein subunit alpha-11, integrin alpha-8, roundabout homolog 1, ADP-ribosylation factor 6, ephrin type-B receptor 2, transforming protein RhoA, proto-oncogene tyrosine-protein kinase Src, synaptobrevin homolog YKT6, Ras-related protein Rab-23, vascular cell adhesion protein 1, lymphocyte function-associated antigen 3, guanine nucleotide-binding protein G (I)/G(S)/G (O) subunit gamma-12, 2′,3′-cyclic-nucleotide 3′-phosphodiesterase, receptor-type tyrosine-protein phosphatase eta, guanine nucleotide-binding protein G(s) subunit alpha isoforms short, ADP-ribosylation factor 5, mitochondrial calcium uniporter regulator 1, nuclear mitotic apparatus protein 1, protein/nucleic acid deglycase DJ-1, V-type immunoglobulin domain-containing suppressor of T-cell activation, CD276, MCAM and Phosphatidylserine synthase 2. The genetically modified cells are also provided. The vesicles find use in methods for reducing inflammatory response in a subject in need thereof.

### Vesicles and Compositions Thereof

Vesicles prepared from cells overexpressing one or more of membrane proteins identified herein as those associated with anti-inflammatory properties of vesicles prepared from human mesenchymal stem cells (MSCs) are disclosed. As explained in the Examples section, EVs, NVs, and SyEVs prepared from MSCs possess anti-inflammatory activity. By comparing the proteome of these three types of vesicles, membrane proteins that are relatively similar in levels in these three types of vesicles were identified. These membrane proteins are highly likely to be instrumental in the imparting the anti-inflammatory properties to these vesicles. Membrane proteins that are less than 1.5 fold different between these three types of vesicles include bifunctional heparan sulfate N-deacetylase/N-sulfotransferase 1, ferroptosis suppressor protein 1, cysteine-rich and transmembrane domain-containing protein, cadherin-6, solute carrier family 22 member 18, promethin, receptor-type tyrosine-protein phosphatase kappa, prolyl endopeptidase FAP, phospholipid-transporting ATPase ID, ADP-ribosyl cyclase/cyclic ADP-ribose hydrolase 2, reticulon-2, ras-related protein Rap-1A, basigin, aldehyde dehydrogenase family 3 member B1, calsyntenin-1, small VCP/p97-interacting protein, GTP-binding protein Rheb, lysophosphatidic acid receptor 1, prolow-density lipoprotein receptor-related protein 1, receptor-type tyrosine-protein phosphatase F, raftlin, guanine nucleotide-binding protein G (q) subunit alpha, guanine nucleotide-binding protein subunit alpha-11, integrin alpha-8, roundabout homolog 1, ADP-ribosylation factor 6, ephrin type-B receptor 2, transforming protein RhoA, proto-oncogene tyrosine-protein kinase Src, synaptobrevin homolog YKT6, Ras-related protein Rab-23, vascular cell adhesion protein 1, lymphocyte function-associated antigen 3, guanine nucleotide-binding protein G (I)/G(S)/G (O) subunit gamma-12, 2′,3′-cyclic-nucleotide 3′-phosphodiesterase, receptor-type tyrosine-protein phosphatase eta, guanine nucleotide-binding protein G(s) subunit alpha isoforms short, ADP-ribosylation factor 5, mitochondrial calcium uniporter regulator 1, nuclear mitotic apparatus protein 1, protein/nucleic acid deglycase DJ-1. In addition, membrane proteins that are present at levels of less than 1.5 fold difference between any two of these three types of vesicles are also highly likely to be required for the anti-inflammatory properties to these vesicles. Such membrane proteins include V-type immunoglobulin domain-containing suppressor of T-cell activation, CD276, MCAM and Phosphatidylserine synthase 2.

In certain aspects, vesicles prepared from a mammalian cell line genetically modified to overexpress a membrane protein selected from the group consisting of: bifunctional heparan sulfate N-deacetylase/N-sulfotransferase 1, ferroptosis suppressor protein 1, cysteine-rich and transmembrane domain-containing protein, cadherin-6, solute carrier family 22 member 18, promethin, receptor-type tyrosine-protein phosphatase kappa, prolyl endopeptidase FAP, phospholipid-transporting ATPase ID, ADP-ribosyl cyclase/cyclic ADP-ribose hydrolase 2, reticulon-2, ras-related protein Rap-1A, basigin, aldehyde dehydrogenase family 3 member B1, calsyntenin-1, small VCP/p97-interacting protein, GTP-binding protein Rheb, lysophosphatidic acid receptor 1, prolow-density lipoprotein receptor-related protein 1, receptor-type tyrosine-protein phosphatase F, raftlin, guanine nucleotide-binding protein G (q) subunit alpha, guanine nucleotide-binding protein subunit alpha-11, integrin alpha-8, roundabout homolog 1, ADP-ribosylation factor 6, ephrin type-B receptor 2, transforming protein RhoA, proto-oncogene tyrosine-protein kinase Src, synaptobrevin homolog YKT6, Ras-related protein Rab-23, vascular cell adhesion protein 1, lymphocyte function-associated antigen 3, guanine nucleotide-binding protein G (I)/G(S)/G (O) subunit gamma-12, 2′,3′-cyclic-nucleotide 3′-phosphodiesterase, receptor-type tyrosine-protein phosphatase eta, guanine nucleotide-binding protein G(s) subunit alpha isoforms short, ADP-ribosylation factor 5, mitochondrial calcium uniporter regulator 1, nuclear mitotic apparatus protein 1, protein/nucleic acid deglycase DJ-1, V-type immunoglobulin domain-containing suppressor of T-cell activation, CD276, MCAM and Phosphatidylserine synthase 2. Also provided is a composition comprising the vesicles; and a pharmaceutically acceptable carrier. The vesicles when administered to a subject reduces the levels of at least one proinflammatory cytokine in the subject.

In certain aspects, the mammalian cell line from which the vesicles are prepared is genetically modified to contain an exogenous nucleic acid sequence encoding the membrane protein. In certain aspects, the exogenous nucleic acid sequence is operably linked to a heterologous promoter. In other aspects, the exogenous nucleic acid sequence is operably linked to an endogenous promoter. In certain aspects, the mammalian cell line from which the vesicles are prepared is genetically modified to increase expression of the endogenous gene encoding the membrane protein. For example, by introduction of a transcriptional activator of the endogenous gene or by inserting a heterologous promoter in the endogenous gene, where the heterologous promoter provides for increased expression of the endogenous gene.

In certain aspects, the mammalian cell line from which the vesicles are produced may be genetically modified to overexpress at least two of the membrane proteins identified herein. In certain aspects, the mammalian cell line from which the vesicles are produced may be genetically modified to overexpress three or more of the membrane proteins.

In certain aspects, the composition comprising the vesicles may include a first population of vesicles prepared from a first mammalian cell line genetically modified to overexpress a first membrane protein and a second population of vesicles prepared from a second mammalian cell line genetically modified to overexpress a second membrane protein, wherein the first and second membrane proteins are independently selected from the group consisting of: bifunctional heparan sulfate N-deacetylase/N-sulfotransferase 1, ferroptosis suppressor protein 1, cysteine-rich and transmembrane domain-containing protein, cadherin-6, solute carrier family 22 member 18, promethin, receptor-type tyrosine-protein phosphatase kappa, prolyl endopeptidase FAP, phospholipid-transporting ATPase ID, ADP-ribosyl cyclase/cyclic ADP-ribose hydrolase 2, reticulon-2, ras-related protein Rap-1A, basigin, aldehyde dehydrogenase family 3 member B1, calsyntenin-1, small VCP/p97-interacting protein, GTP-binding protein Rheb, lysophosphatidic acid receptor 1, prolow-density lipoprotein receptor-related protein 1, receptor-type tyrosine-protein phosphatase F, raftlin, guanine nucleotide-binding protein G (q) subunit alpha, guanine nucleotide-binding protein subunit alpha-11, integrin alpha-8, roundabout homolog 1, ADP-ribosylation factor 6, ephrin type-B receptor 2, transforming protein RhoA, proto-oncogene tyrosine-protein kinase Src, synaptobrevin homolog YKT6, Ras-related protein Rab-23, vascular cell adhesion protein 1, lymphocyte function-associated antigen 3, guanine nucleotide-binding protein G (I)/G(S)/G (O) subunit gamma-12, 2′,3′-cyclic-nucleotide 3′-phosphodiesterase, receptor-type tyrosine-protein phosphatase eta, guanine nucleotide-binding protein G(s) subunit alpha isoforms short, ADP-ribosylation factor 5, mitochondrial calcium uniporter regulator 1, nuclear mitotic apparatus protein 1, protein/nucleic acid deglycase DJ-1, V-type immunoglobulin domain-containing suppressor of T-cell activation, CD276, MCAM and Phosphatidylserine synthase 2. In certain aspects, the composition may further include a third population of vesicles prepared from a third mammalian cell line genetically modified to overexpress a third membrane protein selected from the listed membrane proteins, where the third protein is different from the first and second proteins. In certain aspects, the composition may further include a fourth population of vesicles prepared from a fourth mammalian cell line genetically modified to overexpress a fourth membrane protein selected from the listed membrane proteins, where the fourth protein is different from the first, second, and third proteins. One or more of the mammalian cell lines for preparing the population of vesicles may further be genetically modified to overexpress more than one of the listed membrane proteins.

The genetically modified mammalian cell line may be a mammalian cell line is generated from a parental cell, where the parental cell is a mammalian cell line or a primary cell. The mammalian cell line may be HEK293 cell line, CHO cell line, or embryonic stem cell line. The primary cell may be a stem cell isolated from a subject. In certain aspects, the primary cell may be autologous to the subject. In certain aspects, the stem cell may be an embryonic stem cell, an induced pluripotent stem cell, a hematopoietic stem cell, a neuronal stem cell, a mesenchymal stem cell, a muscle stem cell, or a skin stem cell.

In certain aspects, a mammalian cell genetically modified to overexpress a membrane protein as disclosed herein may express the protein at a level that is 1.5, 2, 2,5 3, 3.5, 4, 4.5, 5, 5.5, 6, 6,5, 7, 7.5, 8, 8.5, 9, 9.5, or 10-fold or greater over the level expressed in the unmodified parental cell from which the genetically modified mammalian cell line was produced.

In certain aspects, vesicles of the present disclosure obtained from a mammalian cell genetically modified to overexpress a membrane protein as disclosed herein contain the membrane protein at a level that is 1.5, 2, 2,5 3, 3.5, 4, 4.5, 5, 5.5, 6, 6,5, 7, 7.5, 8, 8.5, 9, 9.5, or 10-fold or greater over the level expressed in the vesicles from unmodified parental cell from which the genetically modified mammalian cell line was produced. For example, SyEVs of the present disclosure express bifunctional heparan sulfate N-deacetylase/N-sulfotransferase 1 at a level that is 1.5, 2, 2,5 3, 3.5, 4, 4.5, 5, 5.5, 6, 6,5, 7, 7.5, 8, 8.5, 9, 9.5, or 10-fold or greater over the level expressed in SyEVs produced from unmodified parental cell from which the genetically modified mammalian cell line was produced.

In certain aspects, vesicles of the present disclosure are roughly spherical and can range in size, e.g., diameter or largest dimension, from 10 nm-200 nm, e.g., 20 nm-200 nm, 30 nm-200 nm, 40 nm-200 nm, 50 nm-200 nm, 30 nm-175 nm, 30 nm-150 nm, 40 nm-175 nm, or 50 nm-150 nm.

A membrane protein expressed by a cell line as disclosed herein may be the wild-type version of the membrane protein or may be a functional variant that retains features of the membrane protein.

For example, a “bifunctional heparan sulfate N-deacetylase/N-sulfotransferase 1” as used herein encompasses naturally-occurring and synthetic (non-naturally occurring) polypeptides which share at least about 60%, 65%, 70%, 75%, 80%, 85%, 90%, 95% or greater sequence identity at the nucleotide or amino acid level with a naturally-occurring bifunctional heparan sulfate N-deacetylase/N-sulfotransferase 1. “Bifunctional heparan sulfate N-deacetylase/N-sulfotransferase 1” also encompasses fusion proteins, e.g., a bifunctional heparan sulfate N-deacetylase/N-sulfotransferase 1 having a heterologous polypeptide at the N- and/or C-terminus. In certain aspects, a naturally occurring bifunctional heparan sulfate N-deacetylase/N-sulfotransferase 1 is a protein identified with the UniProt Acc. No. P52848.

A “Ferroptosis suppressor protein 1” as used herein encompasses naturally-occurring and synthetic (non-naturally occurring) polypeptides which share at least about 60%, 65%, 70%, 75%, 80%, 85%, 90%, 95% or greater sequence identity at the nucleotide or amino acid level with a naturally-occurring Ferroptosis suppressor protein 1.

“Ferroptosis suppressor protein 1” also encompasses fusion proteins, e.g., a bifunctional heparan sulfate N-deacetylase/N-sulfotransferase 1 having a heterologous polypeptide at the N- and/or C-terminus. In certain aspects, a naturally occurring Ferroptosis suppressor protein 1 is a protein identified with the UniProt Acc. No. Q9BRQ8.

A “Cysteine-rich and transmembrane domain-containing protein 1” as used herein encompasses naturally-occurring and synthetic (non-naturally occurring) polypeptides which share at least about 60%, 65%, 70%, 75%, 80%, 85%, 90%, 95% or greater sequence identity at the nucleotide or amino acid level with a naturally-occurring Cysteine-rich and transmembrane domain-containing protein 1. “Cysteine-rich and transmembrane domain-containing protein 1” also encompasses fusion proteins, e.g., a bifunctional heparan sulfate N-deacetylase/N-sulfotransferase 1 having a heterologous polypeptide at the N- and/or C-terminus. In certain aspects, a naturally occurring Cysteine-rich and transmembrane domain-containing protein 1 is a protein identified with the UniProt Acc. No. Q9H1C7.

A “Cadherin-6” as used herein encompasses naturally-occurring and synthetic (non-naturally occurring) polypeptides which share at least about 60%, 65%, 70%, 75%, 80%, 85%, 90%, 95% or greater sequence identity at the nucleotide or amino acid level with a naturally-occurring Cadherin-6. “Cadherin-6” also encompasses fusion proteins, e.g., a Cadherin-6 having a heterologous polypeptide at the N- and/or C-terminus. In certain aspects, a naturally occurring Cadherin-6 is a protein identified with the UniProt Acc. No. P55285.

A “solute carrier family 22 member 18” as used herein encompasses naturally-occurring and synthetic (non-naturally occurring) polypeptides which share at least about 60%, 65%, 70%, 75%, 80%, 85%, 90%, 95% or greater sequence identity at the nucleotide or amino acid level with a naturally-occurring solute carrier family 22 member 18. “Solute carrier family 22 member 18” also encompasses fusion proteins, e.g., a solute carrier family 22 member 18 having a heterologous polypeptide at the N- and/or C-terminus. In certain aspects, a naturally occurring solute carrier family 22 member 18 is a protein identified with the UniProt Acc. No. Q96BI1.

A “Lipid droplet assembly factor 1” as used herein encompasses naturally-occurring and synthetic (non-naturally occurring) polypeptides which share at least about 60%, 65%, 70%, 75%, 80%, 85%, 90%, 95% or greater sequence identity at the nucleotide or amino acid level with a naturally-occurring Lipid droplet assembly factor 1. “Lipid droplet assembly factor 1” also encompasses fusion proteins, e.g., a Lipid droplet assembly factor 1 having a heterologous polypeptide at the N- and/or C-terminus. In certain aspects, a naturally occurring Lipid droplet assembly factor 1 is a protein identified with the UniProt Acc. No. Q96B96.

A “MAP kinase-activating death domain Protein” as used herein encompasses naturally-occurring and synthetic (non-naturally occurring) polypeptides which share at least about 60%, 65%, 70%, 75%, 80%, 85%, 90%, 95% or greater sequence identity at the nucleotide or amino acid level with a naturally-occurring MAP kinase-activating death domain Protein. “MAP kinase-activating death domain Protein” also encompasses fusion proteins, e.g., a MAP kinase-activating death domain Protein having a heterologous polypeptide at the N- and/or C-terminus. In certain aspects, a naturally occurring MAP kinase-activating death domain Protein is a protein identified with the UniProt Acc. No. Q8WXG6.

A “receptor-type tyrosine-protein phosphatase kappa” as used herein encompasses naturally-occurring and synthetic (non-naturally occurring) polypeptides which share at least about 60%, 65%, 70%, 75%, 80%, 85%, 90%, 95% or greater sequence identity at the nucleotide or amino acid level with a naturally-occurring receptor-type tyrosine-protein phosphatase kappa. “Receptor-type tyrosine-protein phosphatase kappa” also encompasses fusion proteins, e.g., a receptor-type tyrosine-protein phosphatase kappa having a heterologous polypeptide at the N- and/or C-terminus. In certain aspects, a naturally occurring receptor-type tyrosine-protein phosphatase kappa is a protein identified with the UniProt Acc. No. Q15262.

A “Prolyl endopeptidase FAP” as used herein encompasses naturally-occurring and synthetic (non-naturally occurring) polypeptides which share at least about 60%, 65%, 70%, 75%, 80%, 85%, 90%, 95% or greater sequence identity at the nucleotide or amino acid level with a naturally-occurring Prolyl endopeptidase FAP. “Prolyl endopeptidase FAP” also encompasses fusion proteins, e.g., a Prolyl endopeptidase FAP having a heterologous polypeptide at the N- and/or C-terminus. In certain aspects, a naturally occurring Prolyl endopeptidase FAP is a protein identified with the UniProt Acc. No. Q12884.

A “Phospholipid-transporting ATPase ID” as used herein encompasses naturally-occurring and synthetic (non-naturally occurring) polypeptides which share at least about 60%, 65%, 70%, 75%, 80%, 85%, 90%, 95% or greater sequence identity at the nucleotide or amino acid level with a naturally-occurring Phospholipid-transporting ATPase ID. “Phospholipid-transporting ATPase ID” also encompasses fusion proteins, e.g., a Phospholipid-transporting ATPase ID having a heterologous polypeptide at the N- and/or C-terminus. In certain aspects, a naturally occurring Phospholipid-transporting ATPase ID is a protein identified with the UniProt Acc. No. P98198.

A “V-type immunoglobulin domain-containing suppressor of T-cell activation” as used herein encompasses naturally-occurring and synthetic (non-naturally occurring) polypeptides which share at least about 60%, 65%, 70%, 75%, 80%, 85%, 90%, 95% or greater sequence identity at the nucleotide or amino acid level with a naturally-occurring V-type immunoglobulin domain-containing suppressor of T-cell activation. “V-type immunoglobulin domain-containing suppressor of T-cell activation” also encompasses fusion proteins, e.g., a V-type immunoglobulin domain-containing suppressor of T-cell activation having a heterologous polypeptide at the N- and/or C-terminus. In certain aspects, a naturally occurring V-type immunoglobulin domain-containing suppressor of T-cell activation is a protein identified with the UniProt Acc. No. Q9H7M9.

A “CD276” as used herein encompasses naturally-occurring and synthetic (non-naturally occurring) polypeptides which share at least about 60%, 65%, 70%, 75%, 80%, 85%, 90%, 95% or greater sequence identity at the nucleotide or amino acid level with a naturally-occurring CD276. “CD276” also encompasses fusion proteins, e.g., a CD276 having a heterologous polypeptide at the N- and/or C-terminus. In certain aspects, a naturally occurring CD276 is a protein identified with the UniProt Acc. No. Q5ZPR3.

A “MCAM” as used herein encompasses naturally-occurring and synthetic (non-naturally occurring) polypeptides which share at least about 60%, 65%, 70%, 75%, 80%, 85%, 90%, 95% or greater sequence identity at the nucleotide or amino acid level with a naturally-occurring MCAM. “MCAM” also encompasses fusion proteins, e.g., a MCAM having a heterologous polypeptide at the N- and/or C-terminus. In certain aspects, a naturally occurring MCAM is a protein identified with the UniProt Acc. No. P43121.

A “Phosphatidylserine synthase 2” as used herein encompasses naturally-occurring and synthetic (non-naturally occurring) polypeptides which share at least about 60%, 65%, 70%, 75%, 80%, 85%, 90%, 95% or greater sequence identity at the nucleotide or amino acid level with a naturally-occurring Phosphatidylserine synthase 2. “Phosphatidylserine synthase 2” also encompasses fusion proteins, e.g., a Phosphatidylserine synthase 2 having a heterologous polypeptide at the N- and/or C-terminus. In certain aspects, a naturally occurring Phosphatidylserine synthase 2 is a protein identified with the UniProt Acc. No. Q9BVG9.

Percent identity between a pair of sequences may be calculated by multiplying the number of matches in the pair by 100 and dividing by the length of the aligned region, including gaps. Identity scoring only counts perfect matches and does not consider the degree of similarity of amino acids to one another. Only internal gaps are included in the length, not gaps at the sequence ends.

Percent Identity=(Matches×100)/Length of aligned region (with gaps)

Guidance for substitutions, insertions, or deletions may be based on alignments of amino acid sequences of proteins from different species or from a consensus sequence based on a plurality of proteins having the same or similar function.

In certain aspects, the compositions comprising the vesicles and a pharmaceutically acceptable carrier is provided. The carrier may be a diluent, vehicle, excipient, salt, buffer, antioxidant (e.g., ascorbic acid and sodium bisulfate), preservative (e.g., benzyl alcohol, methyl parabens, ethyl or n-propyl, p-hydroxybenzoate), emulsifying agent, suspending agent, dispersing agent, solvent, filler, bulking agent, detergent, and/or adjuvant. For example, a suitable carrier may be physiological saline solution or buffered saline, possibly supplemented with other materials common in pharmaceutical compositions for, e.g., parenteral administration. Neutral buffered saline or saline mixed with serum albumin are further exemplary vehicles. Those skilled in the art will readily recognize a variety of buffers that could be used in the compositions. Typical buffers include, but are not limited to, pharmaceutically acceptable weak acids, weak bases, or mixtures thereof. As an example, the buffer components can be water soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a Tris buffer, N-(2-Hydroxyethyl) piperazine-N′-(2-ethanesulfonic acid) (HEPES), 2-(N-Morpholino) ethanesulfonic acid (MES), 2-(N-Morpholino) ethanesulfonic acid sodium salt (MES), 3-(N-Morpholino) propanesulfonic acid (MOPS), and N-tris [Hydroxymethyl]methyl-3-aminopropanesulfonic acid (TAPS). In certain aspects, an adjuvant included in the disclosed compositions may be poly-ICLC, 1018 ISS, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, JuvImmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PEPTEL, vector system, PLGA microparticles, resiquimod, SRL172, Virosomes and other Virus-like particles, YF-17D, VEGF trap, R848, beta-glucan, Pam3Cys, acrylic or methacrylic polymers, or copolymers of maleic anhydride and Aquila's QS21 stimulon.

Also provided herein are genetically modified mammalian cell lines as disclosed above. In certain aspects, the mammalian cell may be an autologous mammalian cell. The autologous cell may be derived from a tissue or organ of the subject. In certain aspects, the mammalian cell may be a heterologous mammalian cell. In certain aspects, the heterologous mammalian cell may be derived from a tissue or organ of a donor, or from a cell line. The tissue or organ from which the mammalian cell is derived may be bone marrow, blood, blood product, adipose tissue, cord blood, fallopian tube, liver, fetal liver or fetal lungs, etc. In certain aspects, the mammalian cell may be monocytes, macrophages, or dendritic cells.

In certain aspects, the mammalian cell may be a stem cell. In certain aspects, the mammalian cell may be a cell line, such as, an embryonic stem cell line, an induced pluripotent stem cell or any other cell line. In certain aspects, the stem cell may be an embryonic stem cell or a somatic stem cell such as those found in children and adults. In certain aspects, the mammalian cell may be a hematopoietic stem cell, a mammary stem cell, an intestinal stem cell, an endothelial stem cell, a neural stem cell, an olfactory stem cell, a neural crest stem cell, or a testicular stem cell. In certain aspects, the mammalian cell may be a mesenchymal stem cell.

In certain aspects, the mammalian cell may be a cell that naturally produces or is further genetically modified to produce one or more therapeutic agents, such as, trophic factors, e.g., CDNF, GDNF, neurturin, IGF1, VEGF, HGF; chaperones, e.g., HSP104, HSP70; ephA4; ephA4 ligands; Poly(A) Binding Protein Nuclear 1 (PABPN1); matrin ubiquilin 2; Zinc finger protein 106 (ZFP106); IRE1a kinase/Rnase; ubiquilins; TANK Binding Kinase 1 (TBK1); MuSK agonist antibodies; Ankyrin Repeat And KH Domain Containing 1 (ANKHD1); affitins; Glycerophosphodiester phosphodiesterase 2 (GDE2); MMIF; SRSF1 nuclear transport; anti-mir155; miRNA 125b; miRNA 31; miRNA-206; miRNA 133b; TREM2 activating antibodies; SARM1 inhibitor; macrophage migration inhibitory factor (MIF); dominant negative NFKB; Muscle-Specific Kinase; siRNA targeting tristetraprolin; PPARy CoActivator 1alpha; Ret Receptor; notch intracellular domain; TGFβ; INFY, etc.

### Vesicle Production

The vesicles of the present disclosure may be any type of vesicles such as EVs which are naturally released from cells or vesicles generated from extruding cells or by opening cells and revesiculating the cell membrane.

In certain aspects, the EVs are isolated from cell culture supernatants of the genetically modified cells provided herein. For example, the cell culture supernatant may be centrifuged to remove cell debris. The resulting supernatant may be ultracentrifuged to separate large and small vesicles. The supernatant may be centrifuged at 16,500×g for about 20 min to collect large vesicles and at 120,000×g for about 2.5 h at 4° C. to collect small vesicles. Small vesicles (e.g., 40 nm-100 nm diameter) may be purified further by using a density gradient centrifugation.

In certain aspects, the NVs and SyEVs may be prepared from the genetically modified cells provided herein by disrupting the mammalian cell to generate vesicles; separating the vesicles based on density and isolating NVs; exposing the isolated nanovesicles to an alkaline pH to open the nanovesicles thereby generating plasma membrane sheets; purifying the plasma membrane sheets; and applying energy to the purified plasma membrane sheets sufficient to convert the plasma membrane sheets into SyEVs.

In certain aspects, the method may include adding a therapeutic agent to a composition comprising the purified membrane sheets and applying energy to the composition sufficient to convert the plasma membrane sheets into SyEVs comprising the therapeutic agent. In certain aspects, the therapeutic agent does not comprise an anti-inflammatory agent. In certain aspects, the SyEVs do not include a substantial amount of an anti-inflammatory agent, for example, the SyEVs may include a trace amount of an anti-inflammatory agent. In certain aspects, a composition comprising the SyEVs is substantially free of an anti-inflammatory agent.

In certain aspects, disrupting the mammalian cell to generate vesicles may involve mechanical, electrical or chemical methods for cytolysis. Examples of techniques for cytolysis include osmosis, electroporation, sonication, homogenization, detergent treatment, freeze-thawing, extrusion, mechanical degradation, and chemical treatment, but are not limited thereto. In certain aspects, the mammalian cell is not disrupted by detergent treatment. In a mechanical degradation method, a solution of mammalian cells is shaken together with metal, ceramic or sufficiently hard plastic balls. In certain aspects, disrupting the mammalian cell may include applying a shear force to the mammalian cell. Shear force may be applied by extruding the mammalian cell. Extrusion may include forcing the mammalian cells through pores smaller than the size of the mammalian cells. In the context of extrusion, mammalian cells may be forced to sequentially pass through a series of filters having decreasing pore sizes. For example, mammalian cells are sequentially passed through three filters with respective pore sizes of 10 μm-+5 μm-+1 μm to form vesicles.

In certain aspects, disrupting the mammalian cell may include applying acoustic energy to the mammalian cell. Acoustic energy may be applied via a sonication device. Sonication conditions may be adjusted for the desired disruptive energy. For example, low temperature, low energy, and/or short duration for sonication may be used when disrupting cell membrane to generate vesicles. Sonication can be performed with different degree of intensity, including low energy sonication over periods of 1 minute to 3 hours. In certain aspects, sonication may be performed using an ultrasonic probe-type device. In certain aspects, an ultrasonic bath may be used for sonication. The duration of sonication may be adjusted based on the type of device being used to perform the sonication. For example, an ultrasonic probe-type device may provide about 1000 times higher energy than an ultrasonic bath. In certain aspects, ultrasonic probe-type device may be used for disrupting the mammalian cell.

Following disruption of the mammalian cells to generate vesicles, such as, vesicles that have the plasma membrane enclosing cytosolic contents, these vesicles may be isolated from any remaining mammalian cells. Separation of these vesicles from mammalian cells may be performed using differences in size, density, buoyancy, etc. In certain aspects, centrifugation (e.g., density gradient centrifugation or density gradient ultracentrifugation) or filtration may be performed to isolate the vesicles. In certain aspects, the vesicles may be purified using density gradient ultracentrifugation, where vesicles present in between 10% and 50% density gradient may be isolated. The vesicles present in between 10% and 50% density gradient are mostly nanometer sized vesicles or nanovesicles.

The isolated nanovesicles may then be exposed to an alkaline solution to open up the nanovesicles which expels the cytoplasmic content of the nanovesicles. In certain aspects, the alkaline solution used for opening the nanovesicles may have a pH of 11-14. An alkaline solution for opening the nanovesicles may be prepared a sodium carbonate (Na₂CO₃), sodium hydroxide (NaOH), ammonia (NH₃), calcium hydroxide (Ca(OH)₂), potassium hydroxide (KOH), sodium hydrogen carbonate (NaHCO₃), or magnesium hydroxide (Mg(OH)₂) solution. The duration of incubation of the nanovesicles in an alkaline solution may be adjusted based on the number of nanovesicles, total volume of the solution, and the like. As used herein the step of incubating or exposing vesicles to an alkaline pH may include using an alkaline solution having a pH of 9-14, e.g., pH of 10-14, pH of 11-14, pH of 12-14, or pH of 13-14.

Plasma membrane sheets generated from opening of nanovesicles may be separated from whole nanovesicles (i.e., unopened) by utilizing any suitable separation method. In certain aspects, purifying the plasma membrane sheets may involve centrifugation, e.g., centrifugation (such as, density gradient centrifugation or density gradient ultracentrifugation), filtration, or another suitable method, such as size exclusion, dialysis, tangential flow filtration and the like. In certain aspects, the plasma membrane sheets may be purified using density gradient ultracentrifugation, where plasma membrane sheets present in between 10% and 30% density gradient may be isolated. The plasma membrane sheets present in between 10% and 30% density gradient are substantially free of nanovesicles.

In certain aspects, the method of generating the SyEVs may be involve applying energy or force to the purified plasma membrane sheets sufficient to convert the plasma membrane sheets into SyEVs. Suitable sources of energy include mild sonication, shear force, acoustic force, freeze-thaw, and the like. In certain aspects, the purified plasma membrane sheets may be sonicated for a duration of time sufficient to convert the plasma membrane sheets into SyEVs. In certain aspects, the purified plasma membrane sheets may be sonicated by applying energy 100-1000 times less than that applied for disrupting mammalian cells. In certain aspects, mild sonication may include using an ultrasonic bath for converting the plasma membrane sheets into SyEVs.

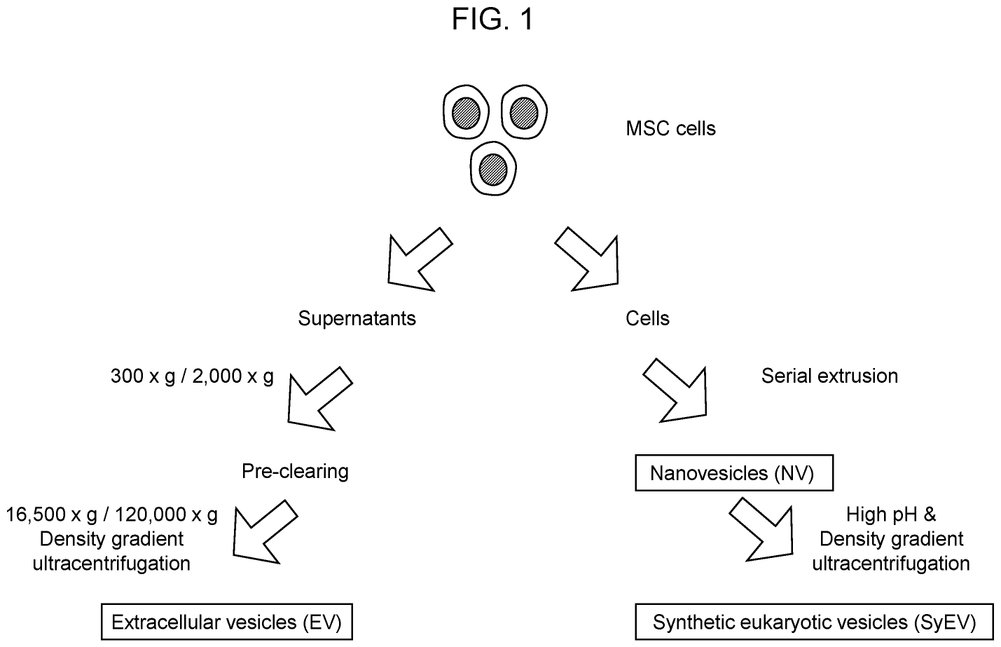

In certain aspects, the vesicles of the present disclosure may be prepared by the method depicted in FIG. 1.

The term “deficient” as used in the context of a component present in the ghost or synthetic nanovesicles (gNVs or SyEVs) derived from a cell as disclosed herein means having at least 50% less of the component, for example, 60%, 70%, 80%, 90%, or 99%, as compared to amount of the component present in non-ghost nanovesicles produced from the same cell. The terms gNVs and SyEVs are used interchangeably herein. Vesicles that have not been prepared by opening and closing of the vesicles are referred to as nanovesicles.

The term “enriched” as used in the context of a protein (e.g., a membrane protein) present in the SyEVs derived from a cell as disclosed herein means that the component makes up a bigger fraction of the total amount of protein in the SyEVs as compared to the fraction of the same protein in NVs produced from the same cell type. For example, the enriched protein may represent at least 25% or more of the total proteins in the SyEVs while the same protein may represent at most 20% of the total proteins in the NVs. An enriched component may be present in the SyEVs at a higher concentration by total weight, e.g., at least a three-fold greater concentration by total weight, e.g., at least 5-fold greater concentration, at least 10-fold greater concentration, at least 30-fold greater concentration, at least 50-fold greater concentration, or at least 100-fold greater concentration than the concentration of that component by total weight in NVs generated from the same cell type from which the SyEVs were derived.

The SyEVs provided herein retain the membrane proteins which membrane proteins are in substantially native conformation. For example, the SyEVs are not exposed to denaturants used as vesiculation agents during generation of the SyEVs. For example, the method for making the SyEVs does not involve a step of exposing the mammalian cell to a vesiculation agent to form vesicles. In other words, the SyEVs are not exposed to vesiculation agents, such as, sulfhydryl blocking agent during or after generation of the SyEVs such that the SyEVs retain the membrane proteins in their native conformation. The SyEVs are not exposed to during or after formation to vesiculation agents such as formaldehyde and dithiothreitol. Sulfhydryl blocking agents include formaldehyde, pyruvic aldehyde, acetaldehyde, glyoxal, glutar aldehyde, acrolein, methacrolein, pyridoxal, N-ethyl malemide (NEM), malemide, chloromercuribenzoate, iodoacetate, potassium arsenite, sodium selenite, thimerosal (merthiolate), benzoyl peroxide, cadmium chloride, hydrogen peroxide, iodosobenzoic acid, meralluride sodium, (mercuhydrin), mercuric chloride, mercurous chloride, chlormerodrin (neohydrin), phenylhydrazine, potassium tellurite, sodium malonate, p-arsenosobenzoic acid, 5,5 ‘-diamino-2, 2’-dimethyl arsenobenzene, N,N′-dimethylene sulfonate disodium salt, iodoacetamide, oxophenarsine (mapharsen), auric chloride, p-chloromercuribenzoic acid, p-chloromercuriphenylsulfonic acid, cupric chloride, iodine merbromin (mercuro chrome) porphyrindine, potassium permanganate, mersalyl(salyrgan), silver nitrate, strong silver protein (protargol), uranyl acetate, etc. Other examples of vesiculation agents include cell toxins such as cytochalasin B or melittin.

The SyEVs may be roughly spherical in shape and may have a diameter smaller than the cells from which the SyEVs are produced. In certain aspects, SyEVs may be relatively large SyEVs that may range in diameter from 100 nm-900 nm, e.g., 100 nm-800 nm, 100 nm-700 nm, 100 nm-600 nm, 100 nm-500 nm, 100 nm-400 nm, 100 nm-300 nm, or 100 nm-200 nm. In certain aspects, SyEVs may be relatively small SyEVs that may range in diameter from 10 nm-100 nm, e.g., 20 nm-100 nm, 30 nm-100 nm, or 40 nm-100 nm. In certain aspects, a preparation of SyEVs, such as a composition of SyEVs may include large and small SyEVs.

SyEVs may be formed by opening nanovesicles (NVs), e.g., by exposing the NVs to high pH, isolating the open sheets of cell membrane, and closing the open sheets of cell membrane to generate the SyEVs. In certain aspects, a gNV may be formed by disrupting the mammalian cell to generate vesicles; separating the vesicles using a density gradient and isolating nanovesicles; exposing the isolated nanovesicles to an alkaline pH to open the nanovesicles thereby generating plasma membrane sheets; purifying the plasma membrane sheets; and applying energy to the purified plasma membrane sheets sufficient to convert the plasma membrane sheets into SyEVs. NVs formed by such a method includes cytoplasmic components, such as, organelles, cytoplasmic proteins, nucleus, nucleic acids (e.g., RNA, such as, mRNA, miRNA, and the like). A gNV is deficient in such components, i.e., has at least 50% less of the component, for example, 60%, 70%, 80%, 90%, or 99% less, as compared to amount of the component present in a NV.

In certain aspects, the SyEVs are made by adding a therapeutic agent to a composition comprising the purified membrane sheets and applying energy to the composition sufficient to convert the plasma membrane sheets into SyEVs comprising the therapeutic agent. In certain aspects, the therapeutic agent does not comprise an anti-inflammatory agent.

In certain aspects, the SyEVs may be loaded with a therapeutic agent that is a nucleic acid, peptide, or protein. The therapeutic agent may be an antibody, a growth factor (e.g., EGF, FGF, VEGF, etc.), siRNA, miRNA, shRNA, etc. In certain aspects, the therapeutic agent may be an anticancer agent or an angiogenesis inhibitor. In certain aspects, the anticancer agent may be DNA alkylating agents, such as mechlorethamine, chlorambucil, phenylalanine, mustard, cyclophosphamide, ifosfamide, carmustine (BCNU), lomustine (CCNU), streptozotocin, busulfan, thiotepa, cisplatin and carboplatin, anti-cancer antibiotics, such as dactinomycin (actinomycin D), doxorubicin (adriamycin), epirubicin, idarubicin, mitoxantrone, plicamycin, mitomycin and C Bleomycin, and plant alkaloids, such as vincristine, vinblastine, paclitaxel, docetaxel, daunorubicin, taxol, oncovin, prednisone, cisplatin, herceptin, rituximab, etoposide, teniposide, topotecan and iridotecan.

### Methods

In certain aspects, a method for treating a subject in need thereof is provided. In certain aspects, a method for reducing inflammation in a subject in need thereof is provided. The method may include administering to the subject an effective amount of the vesicles disclosed herein, wherein the vesicles reduce the levels of at least one proinflammatory cytokine in the subject.

The term “reduced” in the context of inflammatory response means production of a lower level of a proinflammatory cytokine upon administration of the vesicles as compared to that produced in absence of the vesicles. In some embodiments, production of cytokines is lowered by at least 5%, for example, at least 10%, at least 20%, at least 30%, at least 40%, at least 50%, at least 60% or more as compared to that produced in absence of administration of vesicles. The at least one proinflammatory cytokine may include one or more of IL-2, IL-4, IL-6, IL-12, IL-12p70, IL-17, tumor necrosis factor alpha (TNF-α), or interferon gamma (IFN-γ). The mammalian cell from which the vesicles are derived may be as described in the preceding section providing description of the vesicles of the present disclosure.

The subject in need of reduction of inflammation may have or may be susceptible to developing an inflammatory related condition. The inflammatory related condition may be cancer, multiple sclerosis, psoriasis, dry eye disease, asthma, sepsis, infection, Rheumatoid arthritis, ulcerative colitis, Crohn's disease, tuberculosis, hepatitis, sinusitis, autoimmune disease, inflammatory bowel disease, pelvic inflammatory disease, ulcers, atherosclerosis, erythema, necrosis, vasculitis, ankylosing spondylitis, connective tissue disease, kidney disease, sarcoidosis, thyroiditis, osteoarthritis, Rheumatism, chronic inflammatory condition, demyelinating polyneuropathy, pancreatitis, psoriatic arthritis, periodontitis, Behcet's disease, sinusitis, polymyalgia rheumatic, nephritis, diverticulitis, granulomatosis with polyangilitis, granuloma, encephalitis, immune-mediated inflammatory disease, esophagitis, gout, uveitis, myopathy, gallbladder disease, periodic fever syndrome, interstitial cystitis, peritonitis, appendicitis, neurodegenerative disease, Parkinson's disease, Alzheimer's, cerebellar ataxias, systemic lupus erythematous, fibromyalgia, diverticulitis, dermatitis, spinobulbar muscular atrophy (SBMA), lysosomal storage diseases, cerebral palsy, glioma, glioblastoma, muscular dystrophy, ataxia telangiectasia (AT), schizophrenia, depression, bipolar disorder, attention deficit disorder, trisomy 21, amyotrophic lateral sclerosis (ALS) and ankylosing spondylitis. In certain aspects, the inflammatory related condition may be asthma. In certain aspects, the inflammatory related condition may be sepsis. In certain aspects, the inflammatory related condition may be infection. In certain aspects, the inflammatory related condition may be bacterial, viral or parasitic infection.

In certain aspects, the method of reducing inflammation may include administering an additional therapeutic agent to the subject. In certain aspects, the additional therapeutic agent is present in the vesicles. In certain aspects, the method comprises administering a composition comprising the additional therapeutic agent and the vesicles. In certain aspects, the method comprises administering the additional therapeutic agent in conjunction with the vesicles, such as, co-administering (as a single composition or administering at substantially the same time) or administering the vesicles and the additional therapeutic agent sequentially.

The therapeutic agent may be a small molecule, a peptide, a nucleic acid, or a polypeptide. The therapeutic agent may be as provided in the preceding section. The therapeutic agent may have a general anti-inflammatory property, or may target different steps of inflammatory pathways in the cell, such as downstream TLR-receptor activation (Myd88 or NFKB), downstream of cytokine receptors, or Stimulator of Interferon Gens (STING) pathways.

### Administration of Vesicles

The present disclosure contemplates the administration of the disclosed compositions in any appropriate manner for prevention and/or treatment of a condition as described herein. Suitable routes of administration include parenteral (e.g., intramuscular, intravenous, intraarterial, subcutaneous (e.g., injection), intraperitoneal, intracisternal, intraarticular, intraperitoneal, intracerebral (intraparenchymal) and intracerebroventricular), oral, nasal, vaginal, sublingual, intraocular, rectal, topical (e.g., transdermal), sublingual and inhalation, as well as injection directly into a diseased tissue, for example a tumor tissue.

In certain aspects, the administering comprises local administration to a target site in the subject. In certain aspects, the target site comprises or is susceptible to developing an inflammatory response. In certain aspects, the target site has an injury. The target site may be adjacent to a site that has injury. The target site may include a site in the central nervous system. The target site may be brain. The target site may have an arterial blockage. In certain aspects, the administering may be intraarterial administering at the site of arterial blockage, e.g., a catheter used for clot retrieval may be used to administer the vesicles after clot retrieval.

In certain aspects, the compositions of vesicles may be injected into or adjacent a tumor. In certain aspects, a composition of an anticancer agent and a composition of vesicles may be administered simultaneously to a subject.

The present disclosure contemplates methods wherein the compositions of the present disclosure is administered to a subject at least twice daily, at least once daily, at least once every 48 hours, at least once every 72 hours, at least once weekly, at least once every 2 weeks, or once monthly.

### Combination Therapy

The present disclosure contemplates the use of the compositions provided herein in combination with one or more active therapeutic agents or other prophylactic or therapeutic modalities. In such combination therapy, the various active agents frequently have different mechanisms of action. Such combination therapy may be especially advantageous by allowing a dose reduction of one or more of the agents, thereby reducing or eliminating the adverse effects associated with one or more of the agents; furthermore, such combination therapy may have a synergistic therapeutic or prophylactic effect on the underlying disease, disorder, or condition.

As used herein, “combination” is meant to include therapies that can be administered separately, for example, formulated separately for separate administration (e.g., as may be provided in a kit), and therapies that can be administered together in a single formulation (i.e., a “co-formulation”).

In certain embodiments, compositions of the present disclosure are administered or applied sequentially, e.g., where one agent is administered prior to one or more other agents. In other embodiments, the compositions are administered simultaneously, e.g., where two or more compositions are administered at or about the same time; the two or more compositions may be present in two or more separate formulations or combined into a single formulation (i.e., a co-formulation). Regardless of whether the two or more compositions are administered sequentially or simultaneously, they are considered to be administered in combination for purposes of the present disclosure.

The compositions of the present disclosure can be used in combination with other agents useful in the treatment, prevention, suppression or amelioration of the diseases, disorders or conditions set forth herein, including those that are normally administered to subjects suffering from inflammation.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. A composition comprising:
vesicles prepared from a mammalian cell line genetically modified to overexpress a membrane protein selected from the group consisting of: bifunctional heparan sulfate N-deacetylase/N-sulfotransferase 1, ferroptosis suppressor protein 1, cysteine-rich and transmembrane domain-containing protein, cadherin-6, solute carrier family 22 member 18, promethin, receptor-type tyrosine-protein phosphatase kappa, prolyl endopeptidase FAP, phospholipid-transporting ATPase ID, ADP-ribosyl cyclase/cyclic ADP-ribose hydrolase 2, reticulon-2, ras-related protein Rap-1A, basigin, aldehyde dehydrogenase family 3 member B1, calsyntenin-1, small VCP/p97-interacting protein, GTP-binding protein Rheb, lysophosphatidic acid receptor 1, prolow-density lipoprotein receptor-related protein 1, receptor-type tyrosine-protein phosphatase F, raftlin, guanine nucleotide-binding protein G (q) subunit alpha, guanine nucleotide-binding protein subunit alpha-11, integrin alpha-8, roundabout homolog 1, ADP-ribosylation factor 6, ephrin type-B receptor 2, transforming protein RhoA, proto-oncogene tyrosine-protein kinase Src, synaptobrevin homolog YKT6, Ras-related protein Rab-23, vascular cell adhesion protein 1, lymphocyte function-associated antigen 3, guanine nucleotide-binding protein G (I)/G(S)/G (O) subunit gamma-12, 2′,3′-cyclic-nucleotide 3′-phosphodiesterase, receptor-type tyrosine-protein phosphatase eta, guanine nucleotide-binding protein G(s) subunit alpha isoforms short, ADP-ribosylation factor 5, mitochondrial calcium uniporter regulator 1, nuclear mitotic apparatus protein 1, protein/nucleic acid deglycase DJ-1, V-type immunoglobulin domain-containing suppressor of T-cell activation, CD276, MCAM and Phosphatidylserine synthase 2; and
a pharmaceutically acceptable carrier,
wherein the vesicles when administered to a subject reduces the levels of at least one proinflammatory cytokine in the subject.

2. The composition of aspect 1, wherein the mammalian cell line is genetically modified to contain an exogenous nucleic acid sequence encoding the membrane protein.

3. The composition of aspect 2, wherein the exogenous nucleic acid sequence is operably linked to a heterologous promoter.

4. The composition of any one of aspects 1-3, wherein the mammalian cell line is genetically modified to overexpress at least two of the membrane proteins.

5. The composition of any one of aspects 1-3, wherein the mammalian cell line is genetically modified to overexpress three or more of the membrane proteins.

6. The composition of any one of aspects 1-5, wherein the composition comprises a first population of vesicles prepared from a first mammalian cell line genetically modified to overexpress a first membrane protein and a second population of vesicles prepared from a second mammalian cell line genetically modified to overexpress a second membrane protein, wherein the first and second membrane proteins are independently selected from the group consisting of: bifunctional heparan sulfate N-deacetylase/N-sulfotransferase 1, ferroptosis suppressor protein 1, cysteine-rich and transmembrane domain-containing protein, cadherin-6, solute carrier family 22 member 18, promethin, receptor-type tyrosine-protein phosphatase kappa, prolyl endopeptidase FAP, phospholipid-transporting ATPase ID, ADP-ribosyl cyclase/cyclic ADP-ribose hydrolase 2, reticulon-2, ras-related protein Rap-1A, basigin, aldehyde dehydrogenase family 3 member B1, calsyntenin-1, small VCP/p97-interacting protein, GTP-binding protein Rheb, lysophosphatidic acid receptor 1, prolow-density lipoprotein receptor-related protein 1, receptor-type tyrosine-protein phosphatase F, raftlin, guanine nucleotide-binding protein G (q) subunit alpha, guanine nucleotide-binding protein subunit alpha-11, integrin alpha-8, roundabout homolog 1, ADP-ribosylation factor 6, ephrin type-B receptor 2, transforming protein RhoA, proto-oncogene tyrosine-protein kinase Src, synaptobrevin homolog YKT6, Ras-related protein Rab-23, vascular cell adhesion protein 1, lymphocyte function-associated antigen 3, guanine nucleotide-binding protein G (I)/G(S)/G (O) subunit gamma-12, 2′,3′-cyclic-nucleotide 3′-phosphodiesterase, receptor-type tyrosine-protein phosphatase eta, guanine nucleotide-binding protein G(s) subunit alpha isoforms short, ADP-ribosylation factor 5, mitochondrial calcium uniporter regulator 1, nuclear mitotic apparatus protein 1, protein/nucleic acid deglycase DJ-1, V-type immunoglobulin domain-containing suppressor of T-cell activation, CD276, MCAM and Phosphatidylserine synthase 2.

7. The composition of aspect 6, further comprising a third population of vesicles prepared from a third mammalian cell line genetically modified to overexpress a third membrane protein selected from the listed membrane proteins.

8. The composition of aspect 6, further comprising a plurality of populations of vesicles prepared from a plurality mammalian cell lines genetically modified to overexpress a membrane protein selected from the listed membrane proteins.

9. The composition of aspect 6, wherein the first and/or the second mammalian cell line is further genetically modified to overexpress a different membrane protein selected from the listed membrane proteins.

10. The composition of any one of aspects 1-9, wherein the mammalian cell line is generated from a parental cell, wherein the parental cell is a mammalian cell line or a primary cell.

11. The composition of aspect 10, wherein the parental cell is a mammalian cell line, wherein the mammalian cell line is HEK293 cell line, CHO cell line, or embryonic stem cell line.

12. The composition of aspect 10, wherein the parental cell is a primary cell, wherein the primary cell is a stem cell isolated from a subject.

13. The composition of aspect 12, wherein the stem cell is an embryonic stem cell, an induced pluripotent stem cell, a hematopoietic stem cell, a neuronal stem cell, a mesenchymal stem cell, a muscle stem cell, or a skin stem cell.

14. The composition of any one of aspects 1-13, wherein the at least one proinflammatory cytokine comprises IL-2, IL-4, IL-6, IL-12, IL-12p70, IL-17, tumor necrosis factor alpha (TNF-α), or interferon gamma (IFN-γ).

15. The composition of any one of aspects 1-14, wherein the vesicles comprise extracellular vesicles (EVs), nanovesicles (NVs), synthetic eukaryotic vesicles (SyEVs), or a mixture of two or more of EVs, NVs, and SyEVs.

16. A mammalian cell, wherein the mammalian cell is genetically modified to overexpress a membrane protein selected from the group consisting of: bifunctional heparan sulfate N-deacetylase/N-sulfotransferase 1, ferroptosis suppressor protein 1, cysteine-rich and transmembrane domain-containing protein, cadherin-6, solute carrier family 22 member 18, promethin, receptor-type tyrosine-protein phosphatase kappa, prolyl endopeptidase FAP, phospholipid-transporting ATPase ID, ADP-ribosyl cyclase/cyclic ADP-ribose hydrolase 2, reticulon-2, ras-related protein Rap-1A, basigin, aldehyde dehydrogenase family 3 member B1, calsyntenin-1, small VCP/p97-interacting protein, GTP-binding protein Rheb, lysophosphatidic acid receptor 1, prolow-density lipoprotein receptor-related protein 1, receptor-type tyrosine-protein phosphatase F, raftlin, guanine nucleotide-binding protein G (q) subunit alpha, guanine nucleotide-binding protein subunit alpha-11, integrin alpha-8, roundabout homolog 1, ADP-ribosylation factor 6, ephrin type-B receptor 2, transforming protein RhoA, proto-oncogene tyrosine-protein kinase Src, synaptobrevin homolog YKT6, Ras-related protein Rab-23, vascular cell adhesion protein 1, lymphocyte function-associated antigen 3, guanine nucleotide-binding protein G (I)/G(S)/G (O) subunit gamma-12, 2′,3′-cyclic-nucleotide 3′-phosphodiesterase, receptor-type tyrosine-protein phosphatase eta, guanine nucleotide-binding protein G(s) subunit alpha isoforms short, ADP-ribosylation factor 5, mitochondrial calcium uniporter regulator 1, nuclear mitotic apparatus protein 1, protein/nucleic acid deglycase DJ-1, V-type immunoglobulin domain-containing suppressor of T-cell activation, CD276, MCAM and Phosphatidylserine synthase 2.

17. The mammalian cell line of aspect 16, wherein the mammalian cell line is genetically modified to contain an exogenous nucleic acid sequence encoding the membrane protein.

18. The mammalian cell line of aspect 17, wherein the exogenous nucleic acid sequence is operably linked to a heterologous promoter.

19. The mammalian cell line of any one of aspects 16-18, wherein the mammalian cell line is genetically modified to overexpress at least two of the membrane proteins.

20. The mammalian cell line of any one of aspects 16-18, wherein the mammalian cell line is genetically modified to overexpress three or more of the membrane proteins.

21. The mammalian cell line of any one of aspects 16-18, wherein the mammalian cell line is generated from a parental cell, wherein the parental cell is a mammalian cell line or a primary cell.

22. The mammalian cell line of aspect 21, wherein the parental cell is a mammalian cell line, wherein the mammalian cell line is HEK293 cell line, CHO cell line, or embryonic stem cell line.

23. The mammalian cell line of aspect 21, wherein the parental cell is a primary cell, wherein the primary cell is a stem cell isolated from a subject.

24. The mammalian cell line of aspect 23, wherein the stem cell is an embryonic stem cell, an induced pluripotent stem cell, a hematopoietic stem cell, a neuronal stem cell, a mesenchymal stem cell, a muscle stem cell, or a skin stem cell.

25. A method for reducing inflammation in a subject in need thereof, the method comprising:
administering to the subject an effective amount of the composition of any one of aspects 1-15,
wherein the administering reduces at least one proinflammatory cytokine in the subject.

26. The method of aspect 25, wherein the subject has or is susceptible to developing an inflammatory related condition selected from the group consisting of cancer, multiple sclerosis, psoriasis, dry eye disease, asthma, sepsis, infection, Rheumatoid arthritis, ulcerative colitis, Crohn's disease, tuberculosis, hepatitis, sinusitis, autoimmune disease, inflammatory bowel disease, pelvic inflammatory disease, ulcers, atherosclerosis, erythema, necrosis, vasculitis, ankylosing spondylitis, connective tissue disease, kidney disease, sarcoidosis, thyroiditis, osteoarthritis, Rheumatism, chronic inflammatory condition, demyelinating polyneuropathy, pancreatitis, psoriatic arthritis, periodontitis, Behcet's disease, sinusitis, polymyalgia rheumatic, nephritis, diverticulitis, granulomatosis with polyangilitis, granuloma, encephalitis, immune-mediated inflammatory disease, esophagitis, gout, uveitis, myopathy, gallbladder disease, periodic fever syndrome, interstitial cystitis, peritonitis, appendicitis, Parkinson's disease, Alzheimer's, systemic lupus erythematous, fibromyalgia, diverticulitis, dermatitis and ankylosing spondylitis.

27. The method of aspect 26, wherein the inflammatory related condition is asthma.

28. The method of aspect 26, wherein the inflammatory related condition is sepsis.

29. The method of aspect 26, wherein the inflammatory related condition is infection.

30. The method of aspect 29, wherein the infection is a bacterial, viral or parasitic infection.

32. The method of any one of aspects 25-30, wherein the method further comprises administering a therapeutic agent to the subject.

33. The method of aspect 32, wherein the method comprises administering the composition and the therapeutic agent to the subject.

34. The method of aspect 32, wherein the method comprises administering the composition to the subject, wherein the vesicles comprise the therapeutic agent.

35. The method of any one of aspects 32-34, wherein the therapeutic agent comprises a small molecule, a peptide, a nucleic acid, or a polypeptide.

36. The method of any one of aspects 32-34, wherein the administering comprises intravenous, subcutaneous, intramuscular, intraperitoneal, intraarterial, intraarticular, intracerebral (intraparenchymal) or intracerebroventricular administration.

37. The method of any one of aspects 32-34, wherein the administering comprises local administration to a target site in the subject.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or see, second(s); min, minute(s); h or hr, hour(s); aa, amino acid(s); kb, kilobase(s); bp, base pair(s); nt, nucleotide(s); i.m., intramuscular (ly); i.p., intraperitoneal (ly); s.c., subcutaneous (ly); and the like.

### Example 1: Anti-Inflammatory Activity of EVs, NVs and SyEVs in Bacterial Outer Membrane Vesicle-Induced Inflammation

### Methods

### Preparation of EVs, NVs, SyEVs

Preparation of EVs: MSC cell-derived supernatants were centrifuged at 300×g for 10 min and 2,000×g for 20 min at 4° C. to remove cell debris. The resulting supernatants were ultracentrifuged at 16,500×g for 20 min and 120,000×g for 2.5 h at 4° C. to collect large and small vesicles, respectively. Only small vesicles were applied to 4 mL of 50% iodixanol, followed by addition of 4 mL of 30% iodixanol and 2 mL of 10% iodixanol to ultracentrifuge tube. The layers formed between 10% and 30% iodixanol after ultracentrifugation at 100,000×g for 2 hours was collected. Finally, the vesicles were considered EVs (FIG. 1).

Preparation of NVs and SyEVs: MSCs were resuspended at a density of 5×10⁶ cells per mL in total 10 mL of phosphate buffered saline. Cell suspensions were passed five times through each of the membrane filters with a pore size of 10 μm, 5 μm and 1 μm, in that order. Respectively 1 and 2 mL of 50 and 10% solution of iodixanol (Axis-Shield PoC AS), followed by 7 mL of the cell suspension effluent from the membrane filter were sequentially added to each 10 mL ultracentrifuge tube. The layers formed between 50% iodixanol and 10% iodixanol after ultracentrifugation at 100,000×g for 2 hours were collected and considered NVs. The NVs were incubated with high pH solution (200 mM Na₂CO₃, pH 14.0) for 1 hour at 25 degrees. The solution was applied to 4 mL of 50% iodixanol, followed by addition of 4 mL of 30% iodixanol and 2 mL of 10% iodixanol to ultracentrifuge tube. The layers formed between 10% and 30% iodixanol after ultracentrifugation at 100,000×g for 2 hours was collected. Finally, the samples were sonicated for 30 min, and considered SyEVs (FIG. 1).

Quantification of vesicles: Protein concentration of vesicles was determined with a Bradford dye assay (Bio-Rad Laboratories). The particle concentration was assessed by ZetaView analyzer (Particle Metrix GmbH). Measurements were assessed in triplicates and each individual data was obtained from two stationary layers with five times measurements in each layer. Sensitivity of camera was configured at 70 in all measurements. Data were analyzed using ZetaView analysis software version 8.2.30.1.

RAW 264.7 cytokines: RAW 264.7 (1×10⁵), a mouse macrophage cell line, were seeded into 24-well plates, and then E. coli outer membrane vesicles (OMV; 100 ng/ml) were treated for 3 hours to induce inflammation. Various concentrations of EVs, NVs or SyEVs (10⁷, 10⁸, 10⁹) were applied to the cells, and the supernatant concentrations of TNF-α and IL-6 at 15 hours later were measured by ELISA kit (R&D systems).

### Results

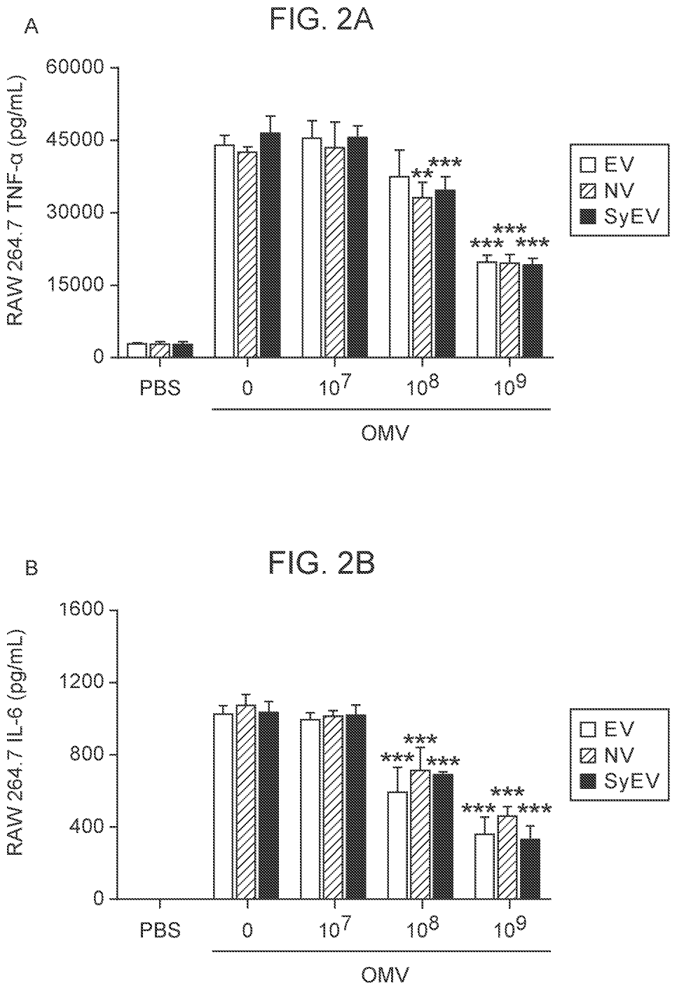

Bacterial OMV have been considered as infectious agents to induce inflammation. SyEVs reduced significantly OMV-induced the release of TNF-α and IL-6 from RAW 264.7 cells comparable to EVs or NVs, suggesting these all vesicles share common proteins related to therapeutic potency (FIG. 2).

### Example 2: Comparative Proteomic Analysis of MSC-Derived EV, NV and SyEV Proteins

### Methods

LC-MS/MS analysis: Two biological replicates of EVs, NVs and SyEVs (30 μg) were digested with trypsin using the filter-aided sample preparation (FASP) method and C18 spin columns desalting according to manufacturer's instructions. All fractions were dried on Speedvac and reconstituted in 3% acetonitrile and 0.2% formic acid and analyzed on Orbitrap Fusion Tribrid mass spectrometer interfaced with Easy-nLC 1200 (Thermo Fisher Scientific). Peptides were trapped on the Acclaim Pepmap 100 C18 trap column (100 μm×2 cm, particle size 5 μm; Thermo Fischer Scientific) and separated on the in-house packed C18 analytical column (75 μm×30 cm, particle size 3 μm) using the gradient from 5% to 32% B in 75 min, from 32% to 100% B in 5 min, solvent A was 0.2% formic acid and solvent B was 80% acetonitrile and 0.2% formic acid. Precursor ion mass spectra were recorded at 120,000 resolutions, the most intense precursor ions were selected, fragmented using collision induced dissociation (CID) at collision energy setting of 35, spectra and the MS/MS spectra were recorded in ion trap with the maximum injection time of 40 ms and the isolation window of 0.7 Da. Charge states 2 to 7 were selected for fragmentation, dynamic exclusion was set to 45 s with 10 ppm tolerance. MS3 spectra for reporter ion quantitation were recorded at 50,000 resolutions with HCD fragmentation at collision energy of 60 using the synchronous precursor selection of the 7 most abundant MS/MS fragments, with the maximum injection time of 100 ms.

Database search: Data analysis was performed using Proteome Discoverer version 2.2 (Thermo Fisher Scientific, Waltham, MA). The database search was performed against the Swissprot Homo sapiens database. Mascot 2.5.1 (Matrix Science) was used as a search engine with precursor mass tolerance of 10 ppm and fragment mass tolerance of 0.6 Da; one missed cleavage was accepted, mono-oxidation on methionine was set as a variable modification, methylthiolation on cysteine was set as a fixed modification. Percolator was used for the validation of identification results with the strict target false discovery rate of 1%, and proteins were only considered when identified in all replicates.

### Results

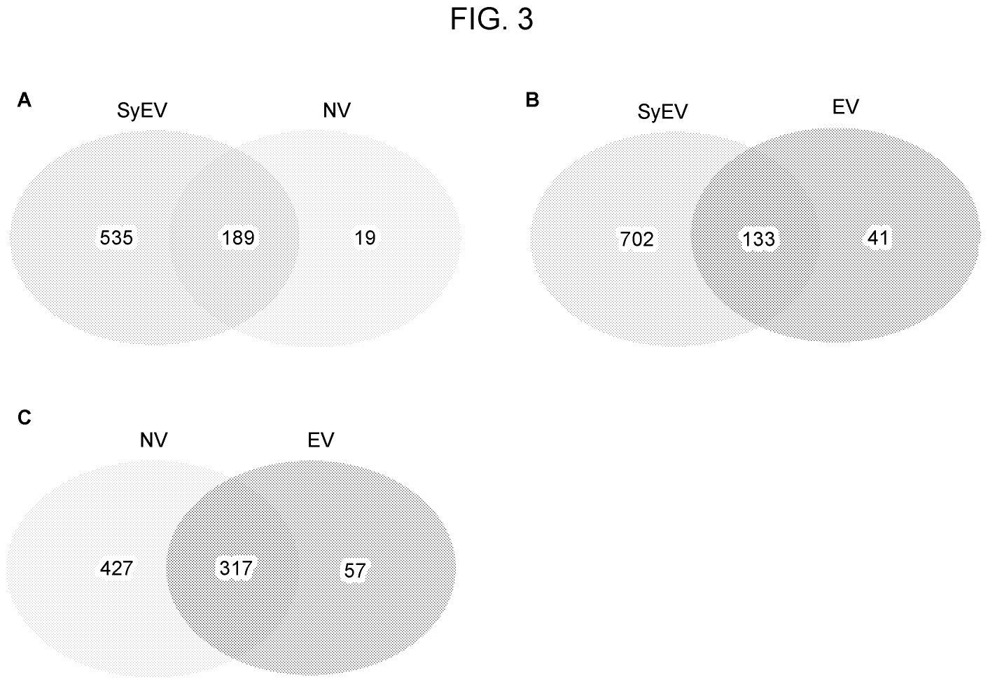

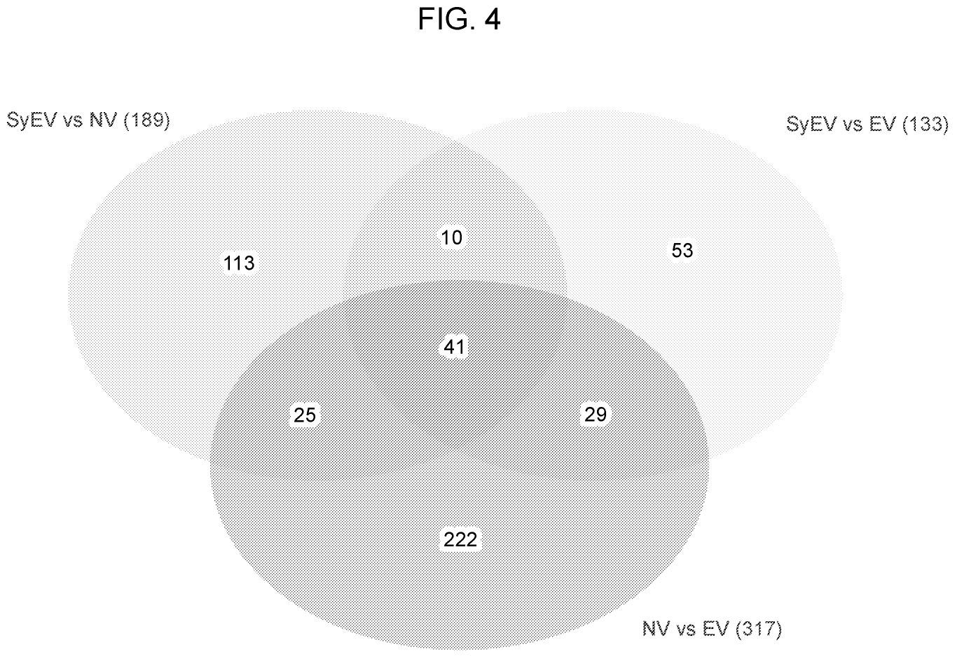

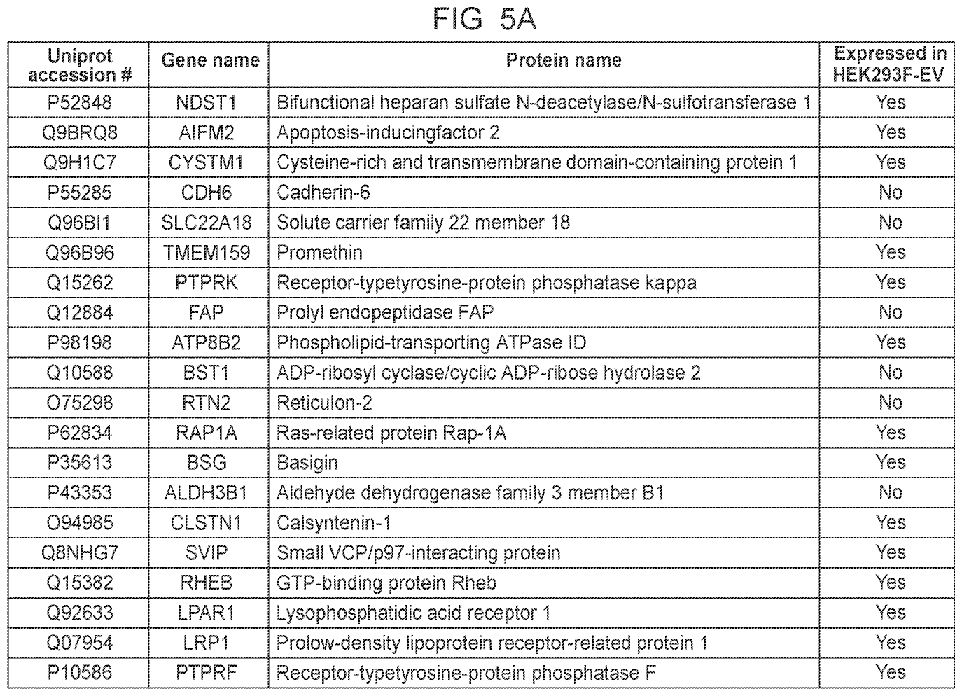

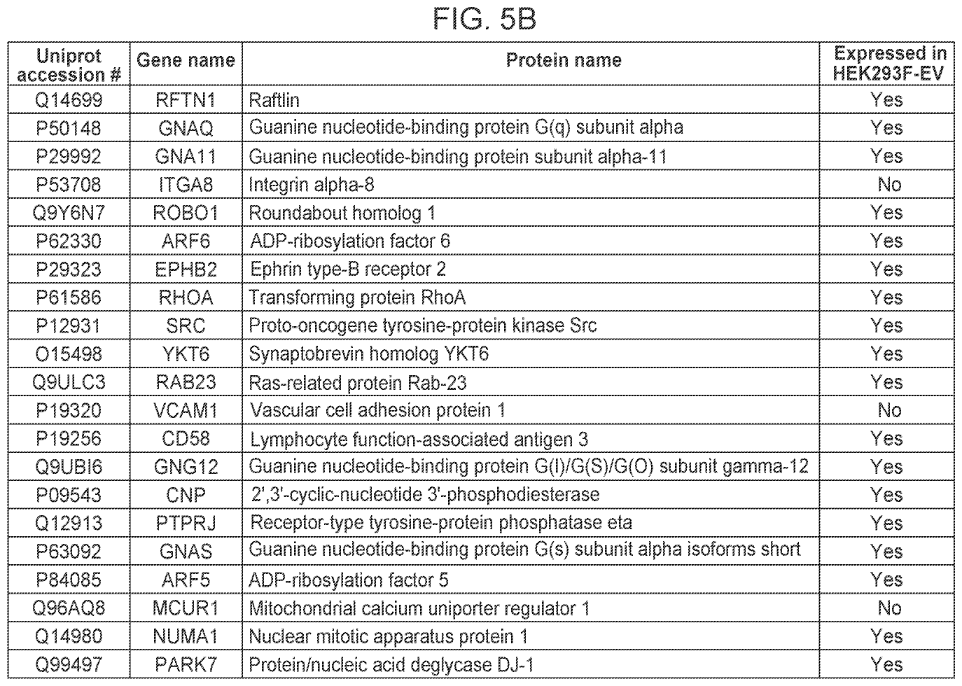

In total, 4686 proteins were identified in the mass spectrometry analysis. Of these 4686 proteins 3661 proteins were quantified in all six samples. Next the Gene ontology (cellular component) for all proteins were downloaded from Uniprot and the GO term “integral component of membrane” was chosen as its definition is “The component of a membrane consisting of the gene products and protein complexes having at least some part of their peptide sequence embedded in the hydrophobic region of the membrane”. This means that the proteins are truly membrane proteins. Out of our 3661 proteins, 605 proteins were associated with this GO term. Next we determined the fold change for these 605 membrane proteins between the three vesicle types. We set the cut off at fold change 1.5. Which means that proteins with a fold change above +1.5 was considered to be upregulated or down regulated, while proteins with a fold change less the 1.5 was considered none-altered between the two vesicle types (FIG. 3). We next compared the proteins considered to be none-altered in the three comparisons, (189 proteins for SyEVs vs NVs, 133 proteins for SyEVs vs EVs and 317 proteins for NVs vs EVs) (FIG. 4). It was then shown that 41 proteins were considered none-altered in all three comparisons and could therefore be considered to have a similar expression in all vesicle types (FIG. 5). Interestingly, bifunctional heparan sulfate N-deacetylase/N-sulfotransferase 1 and ferroptosis suppressor protein 1 are known to be associated with immunosuppression and regulation of repair process (Cell Metab. 2014 Nov. 4;20 (5): 813-826; Nature. 2019 November;575 (7784): 693-698). Furthermore 4, 14 and 9 proteins where none-altered in two out of three comparisons. These could potential also (together with the 10 proteins) be the proteins responsible for the anti-inflammatory effect observed in FIG. 2. Especially, V-type immunoglobulin domain-containing suppressor of T-cell activation, CD276, MCAM and Phosphatidylserine synthase 2 have shown to be involved in immunomodulation and anti-oxidative stress process (Front Immunol. 2020 Oct. 15; 11:580187; Nat Immunol. 2003 September;4 (9): 899-906; Stem Cells. 2020 August;38 (8): 1034-1049; Cell Death Differ. 2016 June;23 (6): 962-78).

What is claimed is:

1. A composition comprising:
vesicles prepared from a mammalian cell line genetically modified to overexpress a membrane protein selected from the group consisting of: bifunctional heparan sulfate N-deacetylase/N-sulfotransferase 1 (NDST1), ferroptosis suppressor protein 1 (AIFM2), cysteine-rich and transmembrane domain-containing protein 1 (CYSTM1), cadherin-6 (CDH6), solute carrier family 22 member 18 (SLC22A18), promethin (TMEM159), receptor-type tyrosine-protein phosphatase kappa (PTPRK), prolyl endopeptidase FAP (FAP), phospholipid-transporting ATPase ID (ATP8B2), ADP-ribosyl cyclase/cyclic ADP-ribose hydrolase 2 (BST1), reticulon-2 (RTN2), ras-related protein Rap-1A (RAP1A), basigin (BSG), aldehyde dehydrogenase family 3 member B1 (ALDH3B1), calsyntenin-1 (CLSTN1), small VCP/p97-interacting protein (SVIP), GTP-binding protein Rheb (RHEB), lysophosphatidic acid receptor 1 (LPAR1), prolow-density lipoprotein receptor-related protein 1 (LRP1), receptor-type tyrosine-protein phosphatase F (PTPRF), raftlin (RFTN1), guanine nucleotide-binding protein G (q) subunit alpha (GNAQ), guanine nucleotide-binding protein subunit alpha-11 (GNA11), integrin alpha-8 (ITGA8), roundabout homolog 1 (ROBO1), ADP-ribosylation factor 6 (ARF6), ephrin type-B receptor 2 (EPHB2), transforming protein RhoA (RHOA), proto-oncogene tyrosine-protein kinase Src (SRC), synaptobrevin homolog YKT6 (YKT6), Ras-related protein Rab-23 (RAB23), vascular cell adhesion protein 1 (VCAM1), lymphocyte function-associated antigen 3 (CD58), guanine nucleotide-binding protein G (I)/G(S)/G (O) subunit gamma-12 (GNG12), 2′,3′-cyclic-nucleotide 3′-phosphodiesterase (CNP), receptor-type tyrosine-protein phosphatase eta (PTPRJ), guanine nucleotide-binding protein G(s) subunit alpha isoforms short (GNAS), ADP-ribosylation factor 5 (ARF5), mitochondrial calcium uniporter regulator 1 (MCUR1), nuclear mitotic apparatus protein 1 (NUMA1), protein/nucleic acid deglycase DJ-1 (PARK7), V-type immunoglobulin domain-containing suppressor of T-cell activation (VISTA), CD276, Cell surface glycoprotein MUC18 (MCAM), and Phosphatidylserine synthase 2 (PTDSS2); and
a pharmaceutically acceptable carrier,
wherein the vesicles when administered to a subject reduces the levels of at least one proinflammatory cytokine in the subject.

2. The composition of claim 1, wherein the mammalian cell line is genetically modified to contain an exogenous nucleic acid sequence encoding the membrane protein.

3. The composition of claim 2, wherein the exogenous nucleic acid sequence is operably linked to a heterologous promoter.

4. The composition of any one of claims 1-3, wherein the mammalian cell line is genetically modified to overexpress at least two of the membrane proteins.

5. The composition of any one of claims 1-3, wherein the mammalian cell line is genetically modified to overexpress three or more of the membrane proteins.

6. The composition of any one of claims 1-5, wherein the composition comprises a first population of vesicles prepared from a first mammalian cell line genetically modified to overexpress a first membrane protein and a second population of vesicles prepared from a second mammalian cell line genetically modified to overexpress a second membrane protein, wherein the first and second membrane proteins are independently selected from the group consisting of: bifunctional heparan sulfate N-deacetylase/N-sulfotransferase 1, ferroptosis suppressor protein 1, cysteine-rich and transmembrane domain-containing protein, cadherin-6, solute carrier family 22 member 18, promethin, receptor-type tyrosine-protein phosphatase kappa, prolyl endopeptidase FAP, phospholipid-transporting ATPase ID, ADP-ribosyl cyclase/cyclic ADP-ribose hydrolase 2, reticulon-2, ras-related protein Rap-1A, basigin, aldehyde dehydrogenase family 3 member B1, calsyntenin-1, small VCP/p97-interacting protein, GTP-binding protein Rheb, lysophosphatidic acid receptor 1, prolow-density lipoprotein receptor-related protein 1, receptor-type tyrosine-protein phosphatase F, raftlin, guanine nucleotide-binding protein G (q) subunit alpha, guanine nucleotide-binding protein subunit alpha-11, integrin alpha-8, roundabout homolog 1, ADP-ribosylation factor 6, ephrin type-B receptor 2, transforming protein RhoA, proto-oncogene tyrosine-protein kinase Src, synaptobrevin homolog YKT6, Ras-related protein Rab-23, vascular cell adhesion protein 1, lymphocyte function-associated antigen 3, guanine nucleotide-binding protein G (I)/G(S)/G (O) subunit gamma-12, 2′,3′-cyclic-nucleotide 3′-phosphodiesterase, receptor-type tyrosine-protein phosphatase eta, guanine nucleotide-binding protein G(s) subunit alpha isoforms short, ADP-ribosylation factor 5, mitochondrial calcium uniporter regulator 1, nuclear mitotic apparatus protein 1, protein/nucleic acid deglycase DJ-1, V-type immunoglobulin domain-containing suppressor of T-cell activation, CD276, MCAM and Phosphatidylserine synthase 2.

7. The composition of claim 6, further comprising a third population of vesicles prepared from a third mammalian cell line genetically modified to overexpress a third membrane protein selected from the listed membrane proteins.

8. The composition of claim 6, further comprising a plurality of populations of vesicles prepared from a plurality mammalian cell lines genetically modified to overexpress a membrane protein selected from the listed membrane proteins.

9. The composition of claim 6, wherein the first and/or the second mammalian cell line is further genetically modified to overexpress a different membrane protein selected from the listed membrane proteins.

10. The composition of any one of claims 1-9, wherein the mammalian cell line is generated from a parental cell, wherein the parental cell is a mammalian cell line or a primary cell.

11. The composition of claim 10, wherein the parental cell is a mammalian cell line, wherein the mammalian cell line is HEK293 cell line, CHO cell line, or embryonic stem cell line.

12. The composition of claim 10, wherein the parental cell is a primary cell, wherein the primary cell is a stem cell isolated from a subject.

13. The composition of claim 12, wherein the stem cell is an embryonic stem cell, an induced pluripotent stem cell, a hematopoietic stem cell, a neuronal stem cell, a mesenchymal stem cell, a muscle stem cell, or a skin stem cell.

14. The composition of any one of claims 1-13, wherein the at least one proinflammatory cytokine comprises IL-2, IL-4, IL-6, IL-12, IL-12p70, IL-17, tumor necrosis factor alpha (TNF-α), or interferon gamma (IFN-γ).

15. The composition of any one of claims 1-14, wherein the vesicles comprise extracellular vesicles (EVs), nanovesicles (NVs), synthetic eukaryotic vesicles (SyEVs), or a mixture of two or more of EVs, NVs, and SyEVs.

16. A mammalian cell, wherein the mammalian cell is genetically modified to overexpress a membrane protein selected from the group consisting of: bifunctional heparan sulfate N-deacetylase/N-sulfotransferase 1, ferroptosis suppressor protein 1, cysteine-rich and transmembrane domain-containing protein, cadherin-6, solute carrier family 22 member 18, promethin, receptor-type tyrosine-protein phosphatase kappa, prolyl endopeptidase FAP, phospholipid-transporting ATPase ID, ADP-ribosyl cyclase/cyclic ADP-ribose hydrolase 2, reticulon-2, ras-related protein Rap-1A, basigin, aldehyde dehydrogenase family 3 member B1, calsyntenin-1, small VCP/p97-interacting protein, GTP-binding protein Rheb, lysophosphatidic acid receptor 1, prolow-density lipoprotein receptor-related protein 1, receptor-type tyrosine-protein phosphatase F, raftlin, guanine nucleotide-binding protein G (q) subunit alpha, guanine nucleotide-binding protein subunit alpha-11, integrin alpha-8, roundabout homolog 1, ADP-ribosylation factor 6, ephrin type-B receptor 2, transforming protein RhoA, proto-oncogene tyrosine-protein kinase Src, synaptobrevin homolog YKT6, Ras-related protein Rab-23, vascular cell adhesion protein 1, lymphocyte function-associated antigen 3, guanine nucleotide-binding protein G (I)/G(S)/G (O) subunit gamma-12, 2′,3′-cyclic-nucleotide 3′-phosphodiesterase, receptor-type tyrosine-protein phosphatase eta, guanine nucleotide-binding protein G(s) subunit alpha isoforms short, ADP-ribosylation factor 5, mitochondrial calcium uniporter regulator 1, nuclear mitotic apparatus protein 1, protein/nucleic acid deglycase DJ-1, V-type immunoglobulin domain-containing suppressor of T-cell activation, CD276, MCAM and Phosphatidylserine synthase 2.

17. The mammalian cell line of claim 16, wherein the mammalian cell line is genetically modified to contain an exogenous nucleic acid sequence encoding the membrane protein.

18. The mammalian cell line of claim 17, wherein the exogenous nucleic acid sequence is operably linked to a heterologous promoter.

19. The mammalian cell line of any one of claims 16-18, wherein the mammalian cell line is genetically modified to overexpress at least two of the membrane proteins.

20. The mammalian cell line of any one of claims 16-18, wherein the mammalian cell line is genetically modified to overexpress three or more of the membrane proteins.

21. The mammalian cell line of any one of claims 16-18, wherein the mammalian cell line is generated from a parental cell, wherein the parental cell is a mammalian cell line or a primary cell.

22. The mammalian cell line of claim 21, wherein the parental cell is a mammalian cell line, wherein the mammalian cell line is HEK293 cell line, CHO cell line, or embryonic stem cell line.

23. The mammalian cell line of claim 21, wherein the parental cell is a primary cell, wherein the primary cell is a stem cell isolated from a subject.

24. The mammalian cell line of claim 23, wherein the stem cell is an embryonic stem cell, an induced pluripotent stem cell, a hematopoietic stem cell, a neuronal stem cell, a mesenchymal stem cell, a muscle stem cell, or a skin stem cell.

25. A method for reducing inflammation in a subject in need thereof, the method comprising:
administering to the subject an effective amount of the composition of any one of claims 1-15,
wherein the administering reduces at least one proinflammatory cytokine in the subject.

26. The method of claim 25, wherein the subject has or is susceptible to developing an inflammatory related condition selected from the group consisting of cancer, multiple sclerosis, psoriasis, dry eye disease, asthma, sepsis, infection, Rheumatoid arthritis, ulcerative colitis, Crohn's disease, tuberculosis, hepatitis, sinusitis, autoimmune disease, inflammatory bowel disease, pelvic inflammatory disease, ulcers, atherosclerosis, erythema, necrosis, vasculitis, ankylosing spondylitis, connective tissue disease, kidney disease, sarcoidosis, thyroiditis, osteoarthritis, Rheumatism, chronic inflammatory condition, demyelinating polyneuropathy, pancreatitis, psoriatic arthritis, periodontitis, Behcet's disease, sinusitis, polymyalgia rheumatic, nephritis, diverticulitis, granulomatosis with polyangilitis, granuloma, encephalitis, immune-mediated inflammatory disease, esophagitis, gout, uveitis, myopathy, gallbladder disease, periodic fever syndrome, interstitial cystitis, peritonitis, appendicitis, Parkinson's disease, Alzheimer's, systemic lupus erythematous, fibromyalgia, diverticulitis, dermatitis and ankylosing spondylitis.

27. The method of claim 26, wherein the inflammatory related condition is inflammatory respiratory disease.

28. The method of claim 26, wherein the inflammatory related condition is sepsis/cytokine storm.

29. The method of claim 26, wherein the inflammatory related condition is infection.

30. The method of claim 29, wherein the infection is a bacterial, viral or parasitic infection.

32. The method of any one of claims 25-30, wherein the method further comprises administering a therapeutic agent to the subject.

33. The method of claim 32, wherein the method comprises administering the composition and the therapeutic agent to the subject.

34. The method of claim 32, wherein the method comprises administering the composition to the subject, wherein the vesicles comprise the therapeutic agent.

35. The method of any one of claims 32-34, wherein the therapeutic agent comprises a small molecule, a peptide, a nucleic acid, or a polypeptide.

36. The method of any one of claims 32-34, wherein the administering comprises intravenous, subcutaneous, intramuscular, intraperitoneal, intraarterial, intraarticular, intracerebral (intraparenchymal) or intracerebroventricular administration.

37. The method of any one of claims 32-34, wherein the administering comprises local administration to a target site in the subject.
